SEQUENCE LISTING (12 sequences)

SEQ ID NO: 1 - DNA, 2350 bp, Corynebacterium glutamicum
  CDS (205)..(2295): tkt
cgggtcagattaagcaaagactactttcggggtagatcacctttgccaaatttgaaccaattaacctaagtcgtagatctgatcatcggatctaacgaaaacgaaccaaaactttggtcccggtttaacccaggaaggattgaccacctaacgaaaacgaaccaaaactttggtcccggtttaacccaggaaggattgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatggtgacctgcaggaaggtgtcacctctgaggcatcctccatcgctggcacccagcagctgggcaacctcatcgtgttctgggatgacaaccgcatctccatcgaagacaacactgagatcgctttcaacgaggacgttgttgctcgttacaaggcttacggctggcagaccattgaggttgaggctggcgaggacgttgcagcaatcgaagctgcagtggctgaggctaagaaggacaccaagcgacctaccttcatccgcgttcgcaccatcatcggcttcccagctccaactatgatgaacaccggtgctgtgcacggtgctgctcttggcgcacgtgaagttgcagcaaccaagactgagcttggattcgatcctgaggctcacttcgcgatcgacgatgaggttatcgctcacacccgctccctcgcagagcgcgctgcacagaagaaggctgcatggcaggtcaagttcgatgagtgggcagctgccaaccctgagaacaaggctctgttcgatcgcctgaactcccgtgagcttccagcgggctacgctgacgagctcccaacatgggatgcagatgagaagggcgtcgcaactcgtaaggcttccgaggctgcacttcaggcactgggcaagacccttcctgagctgtggggcggttccgctgacctcgcaggttccaacaacaccgtgatcaagggctccccttccttcggccctgagtccatctccaccgagacctggtctgctgagccttacggccgtaacctgcacttcggtatccgtgagcacgctatgggatccatcctcaacggcatttccctccacggtggcacccgcccatacggcggaaccttcctcatcttctccgactacatgcgtcctgcagttcgtcttgcagctctcatggagaccgacgcttactacgtctggacccacgactccatcggtctgggcgaagatggcccaacccaccagcctgttgaaaccttggctgcactgcgcgccatcccaggtctgtccgtcctgcgtcctgcagatgcgaacgagaccgcccaggcttgggctgcagcacttgagtacaaggaaggccctaagggtcttgcactgacccgccagaacgttcctgttctggaaggcaccaaggagaaggctgctgaaggcgttcgccgcggtggctacgtcctggttgagggttccaaggaaaccccagatgtgatcctcatgggctccggctccgaggttcagcttgcagttaacgctgcgaaggctctggaagctgagggcgttgcagctcgcgttgtttccgttccttgcatggattggttccaggagcaggacgcagagtacatcgagtccgttctgcctgcagctgtgaccgctcgtgtgtctgttgaagctggcatcgcaatgccttggtaccgcttcttgggcacccagggccgtgctgtctcccttgagcacttcggtgcttctgcggattaccagaccctgtttgagaagttcggcatcaccaccgatgcagtcgtggcagcggccaaggactccattaacggttaattgccctgctgtttttagcttcaacccggggcaatatgattctccggaattt

SEQ ID NO: 2 - protein, 697 aa, Corynebacterium glutamicum
MTLSPELQALTVRNYPSDWSDVDTKAVDTVRVLAADAVENCGSGHPGTAMSLAPLAYTLYQRVMNVDPQDTNWAGRDRFVLSCGHSSLTQYIQLYLGGFGLEMDDLKALRTWDSLTPGHPEYRHTKGVEITTGPLGQGLASAVGMAMAARRERGLFDPTAAEGESPFDHHIYVIASDGDLQEGVTSEASSIAGTQQLGNLIVFWDDNRISIEDNTEIAFNEDVVARYKAYGWQTIEVEAGEDVAAIEAAVAEAKKDTKRPTFIRVRTIIGFPAPTMMNTGAVHGAALGAREVAATKTELGFDPEAHFAIDDEVIAHTRSLAERAAQKKAAWQVKFDEWAAANPENKALFDRLNSRELPAGYADELPTWDADEKGVATRKASEAALQALGKTLPELWGGSADLAGSNNTVIKGSPSFGPESISTETWSAEPYGRNLHFGIREHAMGSILNGISLHGGTRPYGGTFLIFSDYMRPAVRLAALMETDAYYVWTHDSIGLGEDGPTHQPVETLAALRAIPGLSVLRPADANETAQAWAAALEYKEGPKGLALTRQNVPVLEGTKEKAAEGVRRGGYVLVEGSKETPDVILMGSGSEVQLAVNAAKALEAEGVAARVVSVPCMDWFQEQDAEYIESVLPAAVTARVSVEAGIAMPWYRFLGTQGRAVSLEHFGASADYQTLFEKFGITTDAVVAAAKDSING

SEQ ID NO: 3 - DNA, 2160 bp, Corynebacterium glutamicum
  CDS (327)..(2063): poxB
ttagaggcgattctgtgaggtcactttttgtggggtcggggtctaaatttggccagttttcgaggcgaccagacaggcgtgcccacgatgtttaaataggcgatcggtgggcatctgtgtttggtttcgacgggctgaaaccaaaccagactgcccagcaacgacggaaatcccaaaagtgggcatccctgtttggtaccgagtacccacccgggcctgaaactccctggcaggcgggcgaagcgtggcaacaactggaatttaagagcacaattgaagtcgcaccaagttaggcaacacaatagccataacgttgaggagttcagatggcacacagctacgcagaacaattaattgacactttggaagctcaaggtgtgaagcgaatttatggtttggtgggtgacagccttaatccgatcgtggatgctgtccgccaatcagatattgagtgggtgcacgttcgaaatgaggaagcggcggcgtttgcagccggtgcggaatcgttgatcactggggagctggcagtatgtgctgcttcttgtggtcctggaaacacacacctgattcagggtctttatgattcgcatcgaaatggtgcgaaggtgttggccatcgctagccatattccgagtgcccagattggttcgacgttcttccaggaaacgcatccggagattttgtttaaggaatgctctggttactgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgcgattggtgcgcaaagtgttgatcgaaaccgccaggtgatcgcgatgtgtggcgatggtggtttgggcatgctgctgggtgagcttctgaccgttaagctgcaccaacttccgctgaaggctgtggtgtttaacaacagttctttgggcatggtgaagttggagatgctcgtggagggacagccagaatttggtactgaccatgaggaagtgaatttcgcagagattgcggcggctgcgggtatcaaatcggtacgcatcaccgatccgaagaaagttcgcgagcagctagctgaggcattggcatatcctggacctgtactgatcgatatcgtcacggatcctaatgcgctgtcgatcccaccaaccatcacgtgggaacaggtcatgggattcagcaaggcggccacccgaaccgtctttggtggaggagtaggagcgatgatcgatctggcccgttcgaacataaggaatattcctactccatgatgattgatacacctgctgttctcattgaccgcgagcgcttaactgccaacatttccaggatggcagctcacgccggtgcccatgagattgccct

SEQ ID NO: 4 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 5 - DNA, 875 bp, Corynebacterium glutamicum
tgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgc

SEQ ID NO: 6 - DNA, 17 bp, Artificial sequence
  Description of artificial sequence M13/pUC forward primer
gtaaaacgacggccagt

SEQ ID NO: 7 - DNA, 17 bp, Artificial sequence
  Description of artificial sequence M13/pUC reverse primer
caggaaacagctatgac

SEQ ID NO: 8 - DNA, 17 bp, Corynebacterium glutamicum
  Internal primer 1
tgcagcaaccaagactg

SEQ ID NO: 9 - DNA, 21 bp, Corynebacterium glutamicum
  tkt fwd. primer
ctgatcatcggatctaacgaa

SEQ ID NO: 10 - DNA, 21 bp, Corynebacterium glutamicum
  tkt rev. primer
attgccccgggttgaagctaa

SEQ ID NO: 11 - DNA, 20 bp, Corynebacterium glutamicum
  Primer poxBint1
tgcgagatggtgaatggtgg

SEQ ID NO: 12 - DNA, 20 bp, Corynebacterium glutamicum
  Primer poxBint2
gcatgaggcaacgcattagc

(19) United States
(12) Patent Application Publication
(10) Pub. No.: US 2003/0109014 A1
(43) Pub. Date: Jun. 12, 2003

(54) Process for the fermentative preparation of L-amino acids with amplification of the tkt gene

(76) Inventors: Kevin Burke, Newcastle (IE); L. K. Dunican, Bushy Park (IE); Rita Duncian, Galway (IE); Ashling McCormack, Athlone (IE); Cliona Stapleton, Roscrea (IE); Bettina Mockel, Bielefeld (DE); Georg Thierbach, Bielefeld (DE)

Correspondence Address: PILLSBURY WINTHROP, LLP, P.O. BOX 10500, MCLEAN, VA, 22102, US

(21) Appl. No.: 10/143,856

(22) Filed: May 14, 2002

Related U.S. Application Data
(63) Continuation-in-part of application No. 09/986,649, filed on Nov. 9, 2001, now abandoned.
Application No. 09/986,649 is a continuation-in-part of application No. 09/528,196, filed on Mar. 17, 2000, now abandoned.

(51) Int. Cl. C12P 13/08; C12N 9/02; C12N 9/10; C07H 21/04; C12P 21/02; C12N 1/21; C12P 13/04

(52) U.S. Cl. 435/115; 435/252.3; 435/69.1; 435/193; 435/189; 435/320.1; 536/23.2; 435/106

(57) ABSTRACT

The invention relates to a process for the preparation of L-amino acids by the fermentation of coryneform bacteria that over-express a gene encoding transketolase.

### CROSS REFERENCE TO RELATED APPLICATION

This application is a continuation-in-part of U.S. application Ser. No. 09/986,649, filed Nov. 9, 2001, which is a continuation-in-part of U.S. application Ser. No. 09/528,196, filed Mar. 17, 2000.

### FIELD OF THE INVENTION

The invention relates to a process for the fermentative preparation of L-lysine, L-threonine and L-isoleucine using coryneform bacteria in which at least the tkt gene is amplified.

### BACKGROUND OF THE INVENTION

L-Lysine, L-threonine and L-isoleucine are used in animal nutrition, in human medicine and in the pharmaceutical industry. These amino acids may be prepared by fermenting strains of coryneform bacteria, in particular Corynebacterium glutamicum. Work is constantly being undertaken to improve the processes by which amino acids are made. Improvements can relate to fermentation measures, such as, for example, the stirring and supply of oxygen, to the composition of the nutrient media (e.g., the sugar concentration during the fermentation,), to the purification of product (e.g. by ion exchange chromatography), or to the intrinsic output of the microorganism itself.

Methods of mutagenesis are often used to improve the output properties of microorganisms. Strains which are resistant to antimetabolites, such as, for example, the threonine analogue α-amino-β-hydroxyvaleric acid (AHV), or which are auxotrophic for metabolites of regulatory importance and produce L-amino acids such as threonine are obtained in this manner. Recombinant DNA methods have also been employed for some years for improving the Corynebacterium glutamicum strains which produce L-amino acid.

### OBJECT OF THE INVENTION

The inventors had the object of providing new fundamentals for improved processes for the fermentative preparation of L-lysine, L-threonine and L-isoleucine with coryneform bacteria.

### DESCRIPTION OF THE INVENTION

L-Lysine, L-threonine and L-isoleucine are used in human medicine and in the pharmaceuticals industry, in the foodstuffs industry and especially in animal nutrition. There is therefore a general interest in providing improved processes for the preparation of these amino acids. Where L-amino acids are mentioned below, this means L-lysine, L-threonine and L-isoleucine.

The invention provides a process for the fermentative preparation of L-amino acids using coryneform bacteria in which the nucleotide sequence which codes for the enzyme transketolase (EC number 2.2.1.1) (tkt gene) is amplified, in particular over-expressed.

The strains employed preferably already produce L-amino acids before amplification of the tkt gene. The term “amplification” in this connection describes the increase in the intracellular activity of one or more enzymes in a microorganism which are coded by the corresponding DNA, for example by increasing the number of copies of the gene or genes, using a potent promoter or using a gene which codes for a corresponding enzyme having a high activity, and optionally combining these measures. In the present invention, the use of endogenous genes is preferred.

By amplification, in particular over-expression, is meant the activity or concentration of the corresponding protein is in general increased by at least 10%, 25%, 50%, 75%, 100%, 150%, 200%, 300%, 400% or 500%, up to a maximum of 1000% or 2000%, depending on the starting microorganism.

The microorganisms which the present invention provides can prepare L-amino acids from glucose, sucrose, lactose, fructose, maltose, molasses, starch, cellulose or from glycerol and ethanol. They are representatives of coryneform bacteria, in particular of the genus Corynebacterium. Of the genus Corynebacterium, there may be mentioned in particular the species Corynebacterium glutamicum, which is known among specialists for its ability to produce L-amino acids.

Suitable strains of the genus Corynebacterium, in particular of the species Corynebacterium glutamicum, are, for example, the following wild-type strains:

Corynebacterium glutamicum ATCC13032

Corynebacterium acetoglutamicum ATCC15806

Corynebacterium acetoacidophilum ATCC13870

Corynebacterium thermoaminogenes FERM BP-1539

Brevibacterium flavum ATCC14067

Brevibacterium lactofermentum ATCC13869

Brevibacterium divaricatum ATCC14020

L-amino acid-producing mutants prepared from the strains listed above may also be used. Examples of such L-threonine-producing strains include:

Corynebacterium glutamicum ATCC21649

Bevibacterium flavum BB69

Brevibacterium flavum DSM5399

Brevibacterium lactofermentum FERM-BP 269

Brevibacterium lactofermentum TBB-10

Examples of L-isoleucine-producing strains include:

Corynebacterium glutamicum ATCC 14309

Corynebacterium glutamicum ATCC 14310

Corynebacterium glutamicum ATCC 14311

Corynebacterium glutamicum ATCC 15168

Corynebacterium ammoniagenes ATCC 6871

Examples of L-lysine-producing strains include:

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464

Corynebacterium glutamicum ATCC13032

Corynebacterium glutamicum DM58-1

Corynebacterium glutamicum DSM12866.

It has been found that coryneform bacteria produce L-amino acids in an improved manner after over-expression of the tkt gene, which codes for transketolase (EC number 2.2.1.1).

The nucleotide sequence of the tkt gene is disclosed under accession number AB023377 in the databank of the European Molecular Biologies Laboratories (EMBL, Heidelberg, Germany). Ikeda, et al. (Appl. Microbiol. Biotech. 51:201-206 (1999)) describe the effect of amplification of the tkt gene on the formation of L-tryptophan, L-tyrosine and L-phenylalanine. The tkt gene described in the text references mentioned can be used according to the invention. Alleles of the tkt gene which result from the degeneracy of the genetic code or due to sense mutations of neutral function can also be used. The DNA sequence of tkt from C. glutamicum is shown herein as SEQ ID NO:1 and the amino acid sequence encoded by this gene is shown as SEQ ID NO:2.

To achieve an amplification (e.g. over-expression), the number of copies of the corresponding genes may be increased, or the promoter and regulation region or the ribosome binding site upstream of the structural gene may be mutated. Expression cassettes which are incorporated upstream of the structural gene act in the same way. Using inducible promoters, it is additionally possible to increase the expression in the course of fermentative L-amino acid formation. The expression is likewise improved prolonging the life m-RNA or by preventing the degradation of the enzyme. The genes or gene constructs are typically either present in plasmids with a varying number of copies, or are integrated and amplified in the chromosome. Alternatively, an over-expression of genes can be achieved by changing the composition of the media and the culture procedure.

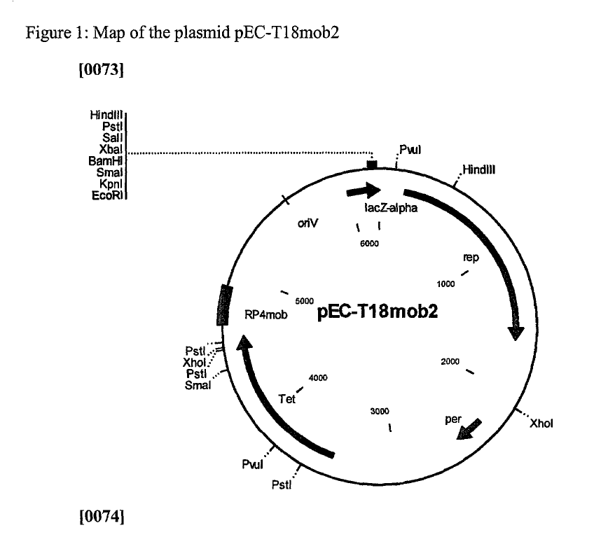

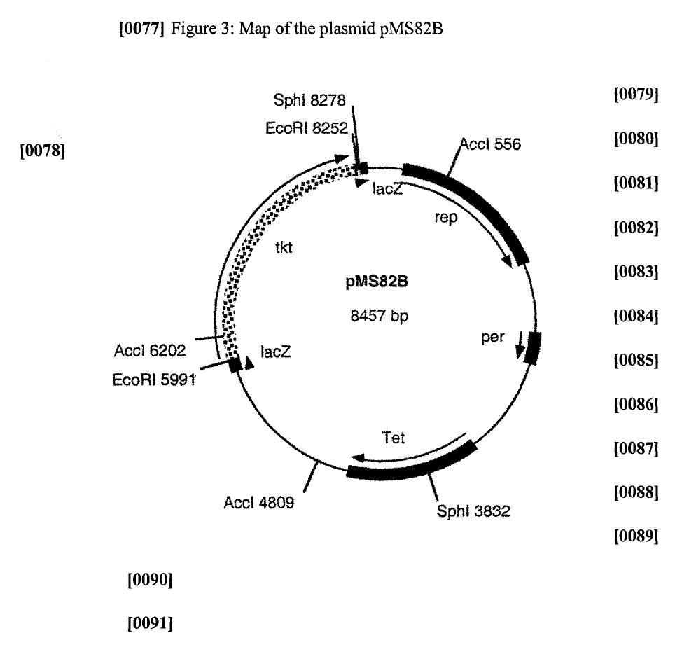

Instructions in this context can be found, inter alia, in Martin, et al. (Bio/Technology 5:137-146 (1987)), in Guerrero, et al. (Gene 138:35-41 (1994)), in Tsuchiya and Morinaga (Bio/Technology 6:428-430 (1988)), in Eikmanns, et al. (Gene 102:93-98 (1991)), in European Patent Specification EP 0 472 869, in U.S. Pat. No. 4,601,893, in Schwarzer and Pühler (Bio/Technology 9:84-87 (1991), in Reinscheid et al. (Appl. Env. Microbiol. 60:126-132 (1994)), in LaBarre, et al. (J. Bacteriol. 175:1001-1007 (1993)), in patent application WO 96/15246, in Malumbres, et al. (Gene 134:15-24 (1993)), in Japanese Laid-Open Specification JP-A-10-229891, in Jensen and Hammer (Biotech. Bioeng. 58:191-195 (1998)) and in known textbooks of genetics and molecular biology. By way of example, transketolase was over-expressed with the aid of a plasmid. The E. coli-C. glutamicum shuttle vector pEC-T18mob2 shown in FIG. 1 was used for this. After incorporation of the tkt gene into pEC-T18mob2 and subsequent orientation correction of the DNA fragment carrying the tkt gene, the plasmid pMS82B shown in FIG. 3 was formed.

Other plasmid vectors which are capable of replication in C. glutamicum, such as e.g., pEKEx1 (Eikmanns et al., Gene 102:93-98 (1991)) or pZ8-1 (EP-B-0 375 889), can be used in the same way. In addition, it may be advantageous for the production of L-amino acids to amplify one or more enzymes of the particular biosynthesis pathway, of glycolysis, of anaplerosis or of amino acid export, in addition to amplification of the tkt gene, which codes for transketolase. Thus, for example, for the preparation of L-threonine, one or more genes chosen from the group consisting of

the hom gene which codes for homoserine dehydrogenase (Peoples et al., Molecular Microbiology 2, 63-72 (1988)) or the hom^{dr} allele which codes for a “feed back resistant” homoserine dehydrogenase (Archer et al., Gene 107, 53-59 (1991),

the gap gene which codes for glyceraldehyde 3-phosphate dehydrogenase (Eikmanns et al., Journal of Bacteriology 174: 6076-6086 (1992)),

the pyc gene which codes for pyruvate carboxylase (Peters-Wendisch et al., Microbiology 144: 915-927 (1998)),

the mqo gene which codes for malate:quinone oxidoreductase (Molenaar et al., European Journal of Biochemistry 254, 395-403 (1998)),

the zwf gene which codes for glucose 6-phosphate dehydrogenase (JP-A-09224661),

the gnd gene which codes for 6-phosphogluconate dehydrogenase (JP-A-9-224662),

the thrE gene which codes for threonine export protein (DE 199 41 478.5; DSM 12840),

the zwa1 gene (DE 199 59 328.0; DSM 13115),

the eno gene which codes for enolase (DE: 19947791.4) can be amplified, in particular over-expressed, at the same time.

For the preparation of L-lysine, one or more genes chosen from the group consisting of:

the dapA gene which codes for dihydrodipicolinate synthase (EP-B 0 197 335),

a lysC gene which codes for a feed back resistant aspartate kinase (Kalinowski et al. (1990), Molecular and General Genetics 224: 317-324),

the gap gene which codes for glycerolaldehyde 3-phosphate dehydrogenase (Eikmanns (1992), Journal of Bacteriology 174:6076-6086),

the pyc gene which codes for pyruvate carboxylase (Eikmanns (1992), Journal of Bacteriology 174:6076-6086),

the mqo gene which codes for malate-quinone oxidoreductase (Molenaar et al., European Journal of Biochemistry 254, 395-403 (1998)),

the zwf gene which codes for glucose 6-phosphate dehydrogenase (JP-A-09224661),

at the same time the gnd gene which codes for 6-phosphogluconate dehydrogenase (JP-A-9-224662),

at the same time the lysE gene which codes for lysine export protein (DE-A-195 48 222)

at the same time the zwa1 gene (DE 199 59 328.0; DSM 13115),

the eno gene which codes for enolase (DE: 19947791.4) can be amplified, preferably over-expressed, at the same time.

It may furthermore be advantageous for the production of L-amino acids at the same time to attenuate one or more of the genes chosen from the group consisting of:

the pck gene which codes for phosphoenol pyruvate carboxykinase (DE 199 50 409.1; DSM 13047),

the pgi gene which codes for glucose 6-phosphate isomerase (U.S. Ser. No. 09/396,478, DSM 12969),

the poxB gene which codes for pyruvate oxidase (DE 199 51 975.7; DSM 13114),

he zwa2 gene (DE: 199 59 327.2; DSM 13113) in addition to the amplification of the tkt gene.

The term “attenuation” means that the activity or concentration of the corresponding protein is in general reduced to 0 to 50%, 0 to 25%, 0 to 10% or 0 to 5% of the activity or concentration of the wild-type protein.

In addition to over-expression of transketolase, it may be advantageous for the production of L-amino acids to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms prepared according to the invention can be cultured continuously or discontinuously in a batch process (batch culture) or in a fed batch (feed process) or in a repeated fed batch process (repetitive feed process) for the purpose of L-amino acid production. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium used must meet the requirements of the particular microorganisms being fermented. Descriptions of culture media for various microorganisms are contained in the handbook “Manual of Methods for General Bacteriology” of the American Society for Bacteriology (Washington D.C., USA, 1981). Sugars and carbohydrates, such as glucose, sucrose, lactose, fructose, maltose, molasses, starch and cellulose, oils and fats, such as soya oil, sunflower oil, groundnut oil and coconut fat, fatty acids, such as palmitic acid, stearic acid and linoleic acid, alcohols, such as glycerol and ethanol, and organic acids, such as acetic acid, can be used as the source of carbon. These substance can be used individually or as a mixture. Organic nitrogen-containing compounds, such as peptones, yeast extract, meat extract, malt extract, corn steep liquor, soya bean flour and urea, or inorganic compounds, such as ammonium sulphate, ammonium chloride, ammonium phosphate, ammonium carbonate and ammonium nitrate, can be used as the source of nitrogen. The sources of nitrogen can be used individually or as a mixture. Potassium dihydrogen phosphate or dipotassium hydrogen phosphate or the corresponding sodium-containing salts can be used as the source of phosphorus. The culture medium must furthermore comprise salts of metals, such as magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances can be added to the culture in the form of a single batch, or can be fed in during the culture in a suitable manner.

Basic compounds, such as sodium hydroxide, potassium hydroxide, ammonia, or acid compounds, such as phosphoric acid or sulfuric acid, can be employed to control the pH. Antifoams, such as fatty acid polyglycol esters, can be employed to control the development of foam. Suitable substances having a selective action, e.g., antibiotics, can be added to the medium to maintain the stability of plasmids. To maintain aerobic conditions, oxygen or oxygen-containing gas mixtures, such as air, are introduced into the culture. The temperature of the culture is usually 20° C. to 45° C., and preferably 25° C. to 40° C. Culturing is continued until a maximum of L-amino acid has formed. This target is usually reached within 10 hours to 160 hours.

The analysis of L-amino acids can be carried out by anion exchange chromatography with subsequent ninhydrin derivatization, as described by Spackman, et al. (Analyt. Chem. 30:1190 (1958)), or it can take place by reversed phase HPLC as described by Lindroth, et al. (Analyt. Chem. 51:1167-1174 (1979)).

The following microorganism has been deposited at the Deutsche Sammlung für Mikrorganismen und Zellkulturen (DSMZ=German Collection of Microorganisms and Cell Cultures, Braunschweig, Germany) in accordance with the Budapest Treaty: Escherichia coli K-12 DH5α/pEC-T18mob2 as DSM 13244.

The following figures are attached:

FIG. 1: Map of the plasmid pEC-T18mob2

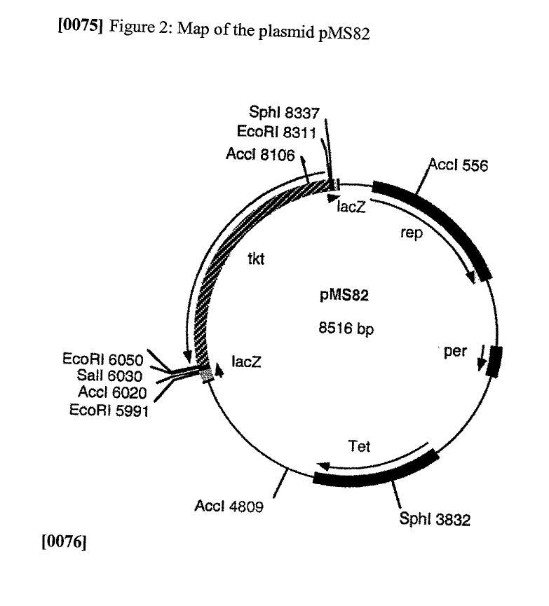

FIG. 2: Map of the plasmid pMS82

FIG. 3: Map of the plasmid pMS82B

The base pair numbers stated are approximate values obtained in the context of reproducibility.

The abbreviations used have the following meaning:

| | |
|---|---|
| Re FIG. 1: | |
| Tet: | Resistance gene for tetracycline |
| oriV: | Plasmid-coded replication origin of E. coli |
| RP4mob: | mob region for mobilizing the plasmid |
| rep: | Plasmid-coded replication origin from C. glutamicum |
| | plasmid pGA1 |
| per: | Gene for controlling the number of copies from pGA1 |
| lacZ-alpha: | lacZα gene fragment (N-terminus) of the β- |
| | galactosidase gene |
| Re FIG. 2 and 3: | |
| Tet: | Resistance gene for tetracycline |
| rep: | Plasmid-coded replication origin from C. glutamicum |
| | plasmid pGA1 |
| per: | Gene for controlling the number of copies from PGA1 |
| lacZ: | Cloning relict of the lacZα gene fragment from pEC- |
| | T18mob2 |
| tkt: | Transketolase gene |

Moreover, the following abbreviations have been used:

| | | |
|---|---|---|
| AccI: | Cleavage site of the restriction enzyme | AccI |
| BamHI: | Cleavage site of the restriction enzyme | BamHI |
| EcoRI: | Cleavage site of the restriction enzyme | EcoRI |
| HindIII: | Cleavage site of the restriction enzyme | HindIII |
| KpnI: | Cleavage site of the restriction enzyme | KpnI |
| PstI: | Cleavage site of the restriction enzyme | PstI |
| PvuI: | Cleavage site of the restriction enzyme | PvuI |
| SalI: | Cleavage site of the restriction enzyme | SalI |
| SacI: | Cleavage site of the restriction enzyme | SacI |
| SmaI: | Cleavage site of the restriction enzyme | SmaI |
| SphI: | Cleavage site of the restriction enzyme | SphI |
| XbaI: | Cleavage site of the restriction enzyme | XbaI |
| XhoI: | Cleavage site of the restriction enzyme | XhoI |

### EXAMPLES

The following examples will further illustrate this invention. The molecular biology techniques, e.g. plasmid DNA isolation, restriction enzyme treatment, ligations, standard transformations of Escherichia coli etc. used are, (unless stated otherwise), described by Sambrook et al., (Molecular Cloning. A Laboratory Manual (1989) Cold Spring Harbour Laboratories, USA).

### Example 1

Construction of a Gene Library of Corynebacterium glutamicum Strain AS019

A DNA library of Corynebacterium glutamicum strain ASO19 (Yoshihama et al., Journal of Bacteriology 162, 591-597 (1985)) was constructed using λ Zap Express™ system, (Short et al., (1988) Nucleic Acids Research, 16: 7583-7600), as described by O'Donohue (O'Donohue, M. (1997). The Cloning and Molecular Analysis of Four Common Aromatic Amino Acid Biosynthetic Genes from Corynebacterium glutamicum. Ph.D. Thesis, National University of Ireland, Galway.). λZap Express™ kit was purchased from Stratagene (Stratagene, 11011 North Torrey Pines Rd., La Jolla, Calif. 92037.) and used according to the manufacturers instructions. AS019-DNA was digested with restriction enzyme Sau3A and ligated to BamHI treated and dephosphorylated λZap Express™ arms.

### Example 2

Cloning and Sequencing of the tkt Gene

1. Cloning

An Escherichia coli strain, AI1118, carrying mutations in the tktA and tktB genes as described by Iida et al., 1993 (Identification and characterization of the tktB gene encoding a second transketolase in Escherichia coli K-12. Journal of Bacteriology 175: 5375-83), was transformed with approx. 500 ng of the AS019 λZap Express™ plasmid library described above. Selection for transformants was made on M9 minimal media, (Sambrook et al (1989). Molecular Cloning. A Laboratory Manual Cold Spring Harbour Laboratories, USA), containing kanamycin at a concentration of 50 mg/l and incubation at 37° C. for 48 hours. Plasmid DNA was isolated from one transformant as according to Birnboim and Doly, 1979, (A rapid alkaline extraction procedure for screening recombinant plasmid DNA. Nucleic Acids Research, 7: 1513-1523.), and designated pTSM2.

2. Sequencing

The clone pTSM2 was commercially sequenced by MWG-Biotech Ltd., Waterside House, Peartree Bridge, Milton Keynes MK6 3BY, U.K. High purity plasmid DNA was prepared for MWG-Biotech, using the QIAprep Spin Miniprep Kit (QIAGEN GmbH, Max-Volmer-Strasse 4, 40724 Hilden, Germany), and subsequently freeze dried using a Lyovac GT 2 freeze dryer (Leybold Heraeus). Initial sequence analysis was carried out using the universal forward and M13 reverse primers.

(SEQ ID NO:6)

| | |
|---|---|
| M13/pUC forward primer: | 5′ GTAAAACGACGGCCAGT 3′ |

(SEQ ID NO:7)

| | |
|---|---|
| M13/pUC reverse primer: | 5′ CAGGAAACAGCTATGAC 3′ |

An internal primer was subsequently designed from the sequence obtained which allowed the entire tkt gene to be deduced. The sequence of the internal primer was as follows:

(SEQ ID NO:8)

| | |
|---|---|
| Internal primer 1: | 5′ TGCAGCAACCAAGACTG 3′ |

Sequence obtained was then analysed using the DNA Strider programme, (Marck (1988), Nucleic Acids Research 16: 1829-1836), version 1.0 on an Apple Macintosh computer. This program allowed for analyses such as restriction site usage, open reading frame analysis and codon usage determination. Searches between DNA sequence obtained and those in EMBL and GenBank databases were achieved using the BLAST programme (Altschul et al., (1997). Nucleic Acids Research, 25: 3389-3402). DNA and protein sequences were aligned using the Clustal V and Clustal W programs (Higgins and Sharp, 1988 Gene 73: 237-244).

The sequence thus obtained is shown in SEQ ID NO 1. The analysis of the nucleotide sequence obtained revealed an open reading frame of 2094 base pairs which was designated as tkt gene. It codes for a protein of 697 amino acids shown in SEQ ID NO 2.

### Example 3

Expression of the tkt Gene

1. Preparation of the Shuttle Vector pEC-T18mob2

The E. coli-C. glutamicum shuttle vector pEC-T18mob2 was constructed according to the prior art. The vector contains the replication region rep of the plasmid pGA1 including the replication effector per (U.S. Pat. No. 5,175,108; Nesvera et al., Journal of Bacteriology 179, 1525-1532 (1997)), the tetracycline resistance-imparting tetA(Z) gene of the plasmid pAG1 (U.S. Pat. No. 5,158,891; gene library entry at the National Center for Biotechnology Information (NCBI, Bethesda, Md., USA) with accession number AF121000), the replication region oriV of the plasmid pMB1 (Sutcliffe, Cold Spring Harbor Symposium on Quantitative Biology 43, 77-90 (1979)), the lacZα gene fragment including the lac promoter and a multiple cloning site (mcs) (Norrander et al. Gene 26, 101-106 (1983)) and the mob region of the plasmid RP4 (Simon et al.,(1983) Bio/Technology 1:784-791).

The vector constructed was transformed in the E. coli strain DH5α (Hanahan, In: DNA cloning. A practical approach. Vol. I. IRL-Press, Oxford, Washington D.C., USA). Selection for plasmid-carrying cells was made by plating-out the transformation batch on LB agar (Sambrook et al., Molecular cloning: a laboratory manual. 2^{nd} Ed. Cold Spring Harbor Laboratory Press, Cold Spring Harbor, N.Y.), which had been supplemented with 5 mg/l tetracycline. Plasmid DNA was isolated from a transformant with the aid of the QIAprep Spin Miniprep Kit from Qiagen and checked by restriction with the restriction enzyme EcoRI and HindIII and subsequent agarose gel electrophoresis (0.8%).

The plasmid was called pEC-T18mob2 and is shown in FIG. 1. It is deposited in the form of the strain Escherichia coli K-12 strain DH5α/pEC-T18mob2 at the Deutsche Sammlung für Mikroorganismen und Zellkulturen (DSMZ=German Collection of Microorganisms and Cell Cultures, Braunschweig, Germany) as DSM 13244.

2. Cloning of the tkt Gene into the E. coli-C. glutamicum Shuttle Vector pEC-T18mob2

PCR was used to amplify DNA fragments containing the entire tkt gene of C. glutamicum and flanking upstream and downstream regions. PCR reactions were carried out using oligonucleotide primers designed from SEQ ID NO 1. Genomic DNA was isolated from Corynebacterium glutamicum ATCC13032 according to Heery and Dunican, (Applied and Environmental Microbiology 59: 791-799 (1993)) and approx. 150-200 ng used as template. The primers used were:

(SEQ ID NO:9)

| | |
|---|---|
| tkt fwd. primer: | 5′ CTG ATC ATC GGA TCT AAC GAA 3′ |

(SEQ ID NO:10)

| | |
|---|---|
| tkt rev. primer: | 5′ ATT GCC CCG GGT TGA AGC TAA 3′ |

PCR parameters were as follows:

35 cycles

95° C. for 6 minutes

94° C. for 1 minute

55° C. for 1 minute

72° C. for 45 seconds

1 mM MgCl₂

The PCR product obtained was cloned into the commercially available pGEM-T vector purchased from Promega Corp. (PGEM-T Easy Vector System 1, cat. no. A1360, Promega UK, Southampton) using E. coli strain JM109 (Yanisch-Perron et al. Gene, 33: 103-119 (1985)) as a host. The entire tkt gene was subsequently isolated from the pGEM T-vector on an SphI/SalI fragment and cloned into the lacZ SphI/SalI region of the E. coli-C. glutamicum shuttle vector pEC-T18mob2 (FIG. 1), and designated pMS82 (FIG. 2). Restriction enzyme analysis with AccI (Boehringer Mannheim GmbH, Germany) revealed the incorrect orientation of the tkt gene in the lacZα gene of pEC-T18mob2. The orientation was corrected by restricting with EcoRI enzyme (Boehringher Mannheim GmbH, Germany) and religating. Restriction enzyme analysis with AccI (Boehringer Mannheim GmbH, Germany) revealed the correct orientation of the tkt gene in the lacZα gene (i.e., downstream the lac-Promoter) of pEC-T18mob2 and this plasmid was designated the name pMS82B (FIG. 3).

### Example 4

Effect of Over-Expression of the tkt Gene in Various Lysine Producers

The L-lysine-producing strain Corynebacterium glutamicum DSM5715 is described in EP-B-0435132 and the strain DSM12866 is described in DE-A-19931314.8. Both strains are deposited at the Deutsche Sammlung für Mikroorganismen und Zellkulturen (German Collection of Microorganisms and Cell Cultures) in Braunschweig (Germany) in accordance with the Budapest Treaty.

1. Preparation of the Strains DSM5715/pMS82B and DSM12866/pMS82B

The strains DSM5715 and DSM12866 were transformed with the plasmid pMS82B using the electroporation method described by Liebl et al. (FEMS Microbiol. Lett. 53:299-303 (1989)). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 5 mg/l tetracycline. Incubation was carried out for 2 days at 33° C.

Plasmid DNA was isolated in each case from a transformant by conventional methods (Peters-Wendisch et al., Microbiol. 144:915-927 (1998)), cleaved with the restriction endonuclease AccI, and the plasmid was checked by subsequent agarose gel electrophoresis. The strains obtained in this way were called DSM5715/pMS82B and DSM12866/pMS82B.

2. Preparation of L-lysine

The Corynebacterium glutamicum strains DSM5715/pMS82B and DSM12866/pMS82B obtained as described above were cultured in a nutrient medium suitable for the production of lysine and the lysine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). The complete medium CgIII was used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH was brought to pH 7.4 | | |

Tetracycline (5 mg/l) was added to this. The preculture was incubated for 16 hours at 33° C. at 240 rpm on a shaking machine. A main culture was seeded from this preculture such that the initial OD (660 nm) of the main culture was 0.1. Medium MM for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) was added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivatization with ninhydrin detection.

The result of the experiment is shown in Table 1.

TABLE 1

| | OD | L-Lysine HCl |
|---|---|---|
| Strain | (660 nm) | g/l |
| DSM5715 | 7.2 | 14.1 |
| DSM5715/pMS82B | 7.2 | 14.8 |
| DSM12866 | 10.9 | 15.3 |
| DSM12866/pMS82B | 11.2 | 16.8 |

### Example 5

Preparation of a Genomic Cosmid Gene Library from Corynebacterium Glutamicum ATCC 13032

Chromosomal DNA from Corynebacterium glutamicum ATCC 13032 was isolated as described by Tauch et al., (Plasmid 33:168-179 (1995)), and partly cleaved with the restriction enzyme Sau3AI (Amersham Pharmacia, Freiburg, Germany, Product Description Sau3AI, Code No. 27-0913-02). The DNA fragments were dephosphorylated with shrimp alkaline phosphatase (Roche Molecular Biochemicals, Mannheim, Germany, Product Description SAP, Code No. 1758250). The DNA of the cosmid vector SuperCos1 (Wahl, et al., Proc. Nat'l Acad. Sci. USA 84:2160-2164 (1987)), obtained from Stratagene (La Jolla, USA, Product Description SuperCos1 Cosmid Vektor Kit, Code No. 251301) was cleaved with the restriction enzyme XbaI (Amersham Pharmacia, Freiburg, Germany, Product Description XbaI, Code No. 27-0948-02) and likewise dephosphorylated with shrimp alkaline phosphatase. The cosmid DNA was then cleaved with the restriction enzyme BamHI (Amersham Pharmacia, Freiburg, Germany, Product Description BamHI, Code no. 27-0868-04).

The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no. 27-0870-04). The ligation mixture was then packed in phages with the aid of Gigapack II XL Packing Extracts (Stratagene, La Jolla, USA, Product Description Gigapack II XL Packing Extract, Code no. 200217). For infection of the E. coli strain NM554 (Raleigh, et al., Nucl. Ac. Res. 16:1563-1575 (1988)) the cells were taken up in 10 mM MgSO₄ and mixed with an aliquot of the phage suspension. The infection and titering of the cosmid library were carried out as described by Sambrook, et al. (Molecular Cloning: A Laboratory Manual, Cold Spring Harbor (1989)), the cells being plated out on LB agar (Lennox, 1955, Virology, 1:190)+100 μg/ml ampicillin. After incubation overnight at 37° C., recombinant individual clones were selected.

### Example 6

Isolation and Sequencing of the poxB Gene

The cosmid DNA of an individual colony (Example 5) was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany).

The DNA of the sequencing vector pZero-1, obtained from Invitrogen (Groningen, Holland, Product Description Zero Background Cloning Kit, Product No. K2500-01) was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (Molecular Cloning: A Laboratory Manual, Cold Spring Harbor (1989)), the DNA mixture being incubated overnight with T4 ligase (Pharmacia Biotech, Freiburg, Germany). This ligation mixture was then electroporated (Tauch, et al., FEMS Microbiol. Lett. 123:343-7 (1994)) into the E. coli strain DH5αMCR (Grant, Proc. Nat'l Acad. Sci. U.S.A. 87:4645-4649 (1990)) and plated out on LB agar (Lennox, Virology, 1:190 (1955)) with 50 μg/ml zeocin.

The plasmid preparation of the recombinant clones was carried out with Biorobot 9600 (Product No. 900200, Qiagen, Hilden, Germany). The sequencing was carried out by the dideoxy chain-stopping method of Sanger, et al. (Proc. Nat'l Acad. Sci. USA 74:5463-5467 (1977)) with modifications according to Zimmermann, et al. (Nuc. Ac. Res. 18:1067 (1990)). The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained was processed using the Staden program package (Nuc. Ac. Res. 14:217-231 (1986)) version 97-0. The individual sequences of the pZero1 derivatives were assembled to a continuous contig. The computer-assisted coding region analysis was prepared with the XNIP program (Staden, Nuc. Ac. Res. 14:217-231 (1986)). Further analyses were carried out with the “BLAST search programs” (Altschul, et al. (Nuc. Ac. Res. 25:3389-3402 (1997)), against the non-redundant databank of the “National Center for Biotechnology Information” (NCBI, Bethesda, Md., USA).

The resulting nucleotide sequence is shown in SEQ ID No:3. Analysis of the nucleotide sequence showed an open reading frame of 1737 base pairs, which was called the poxB gene. The poxB gene codes for a polypeptide of 579 amino acids (SEQ ID NO:4).

### Example 7

Preparation of an Integration Vector for Integration Mutagenesis of the poxB Gene

From the strain ATCC 13032, chromosomal DNA was isolated by the method of Eikmanns, et al. (Microbiol. 140:1817-1828 (1994)). On the basis of the sequence of the poxB gene known for C. glutamicum from Example 8, the following oligonucleotides were chosen for the polymerase chain reaction:

poxBint1:

| | |
|---|---|
| 5′ TGC GAG ATG GTG AAT GGT GG 3′ | (SEQ ID NO:11) |
| poxBint2: | |
| 5′ GCA TGA GGC AAC GCA TTA GC 3′ | (SEQ ID NO:12) |

Integration Mutagenesis of the poxB Gene in the Lysine Producer DSM 5715

The vector pCR2.1poxBint mentioned in Example 7, was electroporated by the method of Tauch et al. (FEMS Microbiol. Lett. 123:343-347 (1994)) in Corynebacterium glutamicum DSM 5715. Strain 5715 is an AEC-resistant lysine producer. The vector pCR2.1poxBint cannot replicate independently in DSM 5715. Selection of clones with pCR2.1poxBint integrated into the chromosome was carried out by plating the electroporation batch on LB agar (Sambrook, et al., Molecular Cloning: A Laboratory Manual, 2^{nd} ed., Cold Spring Harbor Laboratory Press, Cold Spring Harbor, N.Y.), which had been supplemented with 15 mg/l kanamycin. For detection of the integration, the poxBint fragment was labelled with the Dig hybridization kit from Boehringer by the method of “The Dig System Users Guide for Filter Hybridization” of Boehringer Mannheim GmbH (Mannheim, Germany, 1993). Chromosomal DNA of a potential integrant was isolated by the method of Eikmanns, et al. (Microbiology 140:1817-1828 (1994)) and in each case cleaved with the restriction enzymes SalI, SacI, and HindIII. The fragments formed were separated by agarose gel electrophoresis and hybridized at 68° C. with the Dig hybridization kit from Boehringer. The plasmid pCR2.1poxBint mentioned in Example 9 has been inserted into the chromosome of DSM5715 within the chromosomal poxB gene. The strain was called DSM5715::pCR2.1poxBint.

### Example 9

Effect of Over-Expression of the tkt Gene with Simultaneous Elimination of the poxb Gene on the Preparation of Lysine

1. Preparation of the Strain DSM5717::pCR2.1poxBint/pMS82B

The strain DSM5715::pCR2.1poxBint was transformed with the plasmid pMS82B using the electroporation method described by Liebl, et al. (FEMS Microbiol Lett. 53:299-303 (1989)). Selection of the transformants took place on LBHIS agar comprising 18.5 g/l brain-heart infusion broth, 0.5M sorbitol, 5 g/l Bacto-tryptone, 2.5 g/l Bacto-yeast extract, 5 g/l NaCl and 18 g/l Bacto-agar, which had been supplemented with 5 mg/l tetracycline and 25 mg/l kanamycin. Incubation was carried out for 2 days at 33° C.

Plasmid DNA was isolated in each case from a transformant by conventional methods (Peters-Wendisch, et al., Microbiology 144:915-927 (1998)), cleaved with the restriction endonuclease ACCI, and the plasmid was checked by subsequent agarose gel electrophoresis. The strain obtained in this way was called DSM5715:pCR2.1poxBint/pMS82B.

2. Preparation of Lysine

The C. glutamicum strain DSM5717::pCR2.1poxBint/pMS82B obtained as described in Example 9.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l) and kanamycin (25 mg/l)) for 24 hours at 33° C. Starting from this agar plate culture, a preculture was seeded (10 ml medium in a 100 ml conical flask). The complete medium Cg III was used as the medium for the preculture.

Medium CgIII

| | | |
|---|---|---|
| NaCl | 2.5 | g/l |
| Bacto-peptone | 10 | g/l |
| Bacto-Yeast Extract | 10 | g/l |
| Glucose (autoclaved separately) | 2% | (w/v) |
| The pH was brought to 7.4 | | |

Tetracycline (5 mg/l) and kanamycin (25 mg/l) were added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 58 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state.

Culturing is carried out in a 10 ml volume in a 100 ml conical flask with baffles. Tetracycline (5 mg/ml) and kanamycin (25 mg/ml) were added. Culturing was carried out at 33° C. and 80% atmospheric humidity. After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronk (Hamburg, Germany) by ion exchange chromatography and post-column derivation with ninhydrin detection. The result of the experiment is shown in Table 2.

TABLE 2

| | OD | L-Lysine |
|---|---|---|
| Strain | (660 nm) | g/l |
| DSM5715 | 10.8 | 15.9 |
| DSM5715::pCR2.1pox | 7.1 | 16.7 |
| Bint | | |
| DSM5715::pCR2.1pox | 7.7 | 17.3 |
| Bint/pMS82B | | |

What is claimed is:

1. A process for the preparation of L-lysine or L-threonine by the fermentation of coryneform bacteria, comprising:
a) fermenting L-lysine or L-threonine producing bacteria in which the endogenous gene coding for transketolase (tkt) is over-expressed; and
b) isolating said L-lysine or said L-threonine from said bacteria or from the medium in which said bacteria are fermented.

2. The process of claim 1, wherein said process is for the preparation of L-lysine and, in addition to over-expressing said endogenous gene coding for tkt, said bacteria have at least one additional endogenous gene that is over-expressed or amplified, said additinal endogenous gene being selected from the group consisting of:
(a) the dapA gene which codes for dihydrodipicolinate synthase;
(b) the lysC gene which codes for a feedback resistant aspartate kinase;
(c) the gap gene which codes for glycerolaldehyde 3-phosphate dehydrogenase;
(d) the pyc gene which codes for pyruvate carboxylase;
(e) the zwf gene which codes for glucose 6-phosphate dehydrogenase;
(f) the gnd gene which codes for 6-phosphogluconate dehydrogenase;
(g) the lysE gene which codes for lysine export protein;
(h) the mqo gene which codes for malate-quinone oxidoreductase; and
the eno gene which codes for enolase.

3. The process of claim 1, wherein said process is for the preparation of L-threonine and, in addition to over-expressing said endogenous gene coding for tkt, said bacteria have at least one additional endogenous gene that is over-expressed or amplified, said additinal endogenous gene being selected from the group consisting of:
the hom gene which codes for homoserine dehydrogenase;
the hom^{dr} allele which codes for a “feed back resistant” homoserine dehydrogenase;
(c) the gap gene which codes for glycerolaldehyde 3-phosphate dehydrogenase;
(d) the pyc gene which codes for pyruvate carboxylase;
(e) the mqo gene which codes for malate:quinone oxidoreductase;
(f) the zwf gene which codes for glucose 6-phosphate dehydrogenase;
(g) the gnd gene which codes for 6-phosphogluconate dehydrogenase;
(h) the thrE gene which codes for threonine export protein; and
(i) the eno gene which codes for enolase.

4. The process of claim 1, wherein said process is for the preparation of L-lysine and, in addition to over-expressing said endogenous gene coding for tkt, said bacteria have at least one endogenous gene that is attenuated, said endogenous gene being selected from the group consisting of:
(a) the pck gene which codes for phosphoenol pyruvate carboxykinase; and
(b) the poxB gene which codes for pyruvate oxidase.
The process of any one of claims 1-4, wherein the over-expression or amplification of said gene coding for tkt or said additional endogenous gene is accomplished by transforming said bacteria with a plasmid vector carrying said gene coding for tkt or said additional endogenous gene.

6. A plasmid vector pEC-T18mob2 deposited under the designation DSM 13244 in K-12 DH5α, shown in FIG. 1.

7. A plasmid vector pEC-T18mob2 as claimed in claim 6, which additionally carries the tkt gene.

8. A coryneform microorganism, in particular of the genus Corynebacterium, transformed by the introduction of the plasmid vector as claimed in claim 10, which additionally contains the tkt gene.
